(19) United States
(12) Patent Application Publication
(10) Pub. No.: US 2012/0195929 A1
(43) Pub. Date: Aug. 2, 2012

(54) COMPOUNDS THAT MODULATE AUTOIMMUNITY AND METHODS OF USING THE SAME

(76) Inventors: George Eisenbarth, Golden, CO (US); Aaron Michels, Aurora, CO (US); David Ostrov, Gainesville, FL (US); Maki Nakayama, Denver, CO (US)

(21) Appl. No.: 13/376,119

(22) PCT Filed: Jun. 4, 2010
(86) PCT No.: PCT/US10/37495

§ 371 Date: Apr. 12, 2012

Related U.S. Application Data
(60) Provisional application No. 61/184,072, filed on Jun. 4, 2009.

(51) Int. Cl. A61K 31/44; A61K 31/663; A61K 31/194; A61K 31/198; A61K 31/351; A61P 3/10; A61K 31/47; A61K 31/4965; A61K 31/661; A61K 31/185; A61K 31/4172; A61P 37/06; A61K 31/662; A61K 31/222

(52) U.S. Cl. 424/278.1

(57) ABSTRACT

The invention provides methods of preventing, treating or ameliorating autoimmune diseases such as diabetes by modulating the binding of MHC class II molecules to antigenic peptides or fragments of antigenic peptides of the autoimmune disease by the administration of small organic compounds. The invention also provides pharmaceutical compositions comprising the therapeutically effective small organic compounds and methods of using the same.

### GOVERNMENT INTEREST

This invention was made with government support by grants from the National Institutes of Diabetes & Digestive & Kidney Diseases (R01 DK055969 and P30 DK057516), the Juvenile Diabetes Foundation (4-2007-1056), the Brehm Coalition, and the Children's Diabetes Foundation. The government has certain rights in this invention.

### TECHNICAL FIELD

The invention relates to therapeutic compounds, pharmaceutical compositions containing the same and their use in the prevention or treatment of autoimmune diseases, such as autoimmune diabetes.

### BACKGROUND OF INVENTION

Autoimmune disorders are diseases caused by the body producing an inappropriate immune response against its own tissues, in which the immune system creates T lymphocytes and autoantibodies that attack one's own cells, tissues, and/or organs. Researchers have identified 80-100 different autoimmune diseases and suspect at least 40 additional diseases have an autoimmune basis.

Autoimmune disorders are classified into two types, organ-specific (directed mainly at one organ) and non-organ-specific (widely spread throughout the body). Examples of organ-specific autoimmune disorders are insulin-dependent Type 1 diabetes, which affects the pancreas, Hashimoto's thyroiditis and Graves' disease, which affects the thyroid gland, pernicious anemia, which affects the stomach, Addison's disease, which affects the adrenal glands, chronic active hepatitis, which affects the liver and myasthenia gravis which, affects the muscles. Examples of non-organ-specific autoimmune disorders are rheumatoid arthritis, multiple sclerosis and lupus.

Autoimmune diseases are often chronic, debilitating and life-threatening. The National Institutes of Health (NIH) estimates up to 23.5 million Americans suffer from autoimmune disease and that the prevalence is rising. It has been estimated that autoimmune diseases are among the ten leading causes of death among women in all age groups up to 65 years. Most autoimmune diseases cannot yet be treated directly, but are treated to alleviate the symptoms associated with the condition. Some of the current treatments include administration of corticosteroid drugs, non-steroidal anti-inflammatory drugs (NSAIDs) or more powerful immunosuppressant drugs such as cyclophosphamide, methotrexate and azathioprine that suppress the immune response and stop the progression of the disease. Radiation of the lymph nodes and plasmapheresis (a procedure that removes the diseased cells and harmful molecules from the blood circulation) are other ways of treating an autoimmune disease. However, these treatments often have devastating long-term side effects.

One of the most prevalent organ-specific autoimmune diseases, Type 1 diabetes, is characterized by the production of autoantibodies that target the insulin-secreting pancreatic beta cells. The destruction of the beta cells is mainly due to the action of T cells. In most cases, T cells can respond to an antigen only when the antigen is properly presented by an antigen presenting cell expressing the appropriate major histocompatibility complex (MHC) molecule. Thus, T cell immune response to an antigen requires recognition by the T cell receptor of an antigen coupled to a MHC molecule, and this recognition requires the assembly of a tri-molecular complex between an antigen, a MHC molecule and T cell receptor. In particular, the recognized peptide (when peptide autoantigen) must be in an appropriate register (or postion along the presenting MHC molecule groove).

Evidence strongly indicates that insulin/proinsulin is a key or primary auto-antigen in the development of type 1 diabetes in the NOD (non-obese diabetic) mouse model. Initial cloning of T cells from islets of NOD mice led to the discovery that the native insulin B chain amino acids 9-23 (B:9-23 insulin peptide) is the dominant antigenic peptide epitope presented by the class II MHC molecule I-A. Mice lacking the native B:9-23 sequence fail to develop diabetes and development of insulin autoantibodies and insulitis are markedly decreased. Restoring the native B:9-23 sequence with an islet transplant (but not bone marrow transplant) or peptide immunization, or a native proinsulin transgene, restores anti-insulin autoimmunity and generates CD4 T cells that cause diabetes.

The major genetic determinant of islet autoimmunity and diabetes in man and animal models are genes within the major histocompatibility complex, and in particular, class II MHC alleles. The NOD mice's unique sequence of IA (homologous to DQ of man) and lack of expression of I-E (shared with many standard mouse strains) are essential for the development of diabetes. The crystal structure of I-A^{g7} bound to the peptide has allowed the modeling of peptide binding to this molecule. Similar modeling has been performed for the human diabetogenic allele/molecule DQ8, which has analogous sequence to I-A^{g7}. Unanue and coworkers have defined two different registers of binding of the B:9-23 peptide to I-A^{g7} and multiple investigators have utilized the B:9-23 peptide for prevention of diabetes (Levisetti M G, Suri A, Petzold S J, and Unanue E R, J. Immunol. 178(10):6051-6057 (2007); Bresson D L von Herrath M, Autoimmun. Rev. 6(5):315-322 (2007); Fukushima K, Abiru N, Nagayama Y et al, Biochem. Biophys. Res. Com. 367(4):719-724, 2008).

There are alternative hypotheses as to why I-A^{g7}(and DQB1*0302 44) is associated with islet autoimmunity. One hypothesis is that the molecule is a poor binder of peptides and potentially unstable, and such instability or defective binding might limit negative selection of autoimmune T cells within the thymus. Another hypothesis is that I-A^{g7} is critical for presentation of specific autoantigenic peptide(s) in the periphery. The second hypothesis is supported by the observation that I-A alleles such as IAk prevent NOD diabetes but enhance alternative autoimmune disorders, suggesting that class II alleles determine the specific organ targeted rather than general susceptibility to autoimmunity.

Thus, there exists a need in the art for safer and more effective methods for treatment and prevention of autoimmune diseases. The instant invention addresses these needs by providing small molecules useful in the treatment and prevention of autoimmune diseases.

### SUMMARY OF INVENTION

The present invention is drawn to molecules that can prevent or enhance the binding of T cell receptors to insulin/proinsulin peptides presented by class II MHC molecules, as well as therapeutic uses of these molecules to prevent or slow the formation of autoimmune diseases, such as diabetes, in a mammal.

Class II major histocompatability molecules are the primary susceptibility locus for many autoimmune diseases, including type 1 diabetes. “Diabetogenic” alleles HLA-DQ8 in humans and I-A^{g7} in non-obese diabetic (NOD) mice confer disease risk, and both molecules share structural similarities. In particular, a polymorphism in pocket 9 of both I-A^{g7} and DQ8 is believed to confer risk due to the substitution of aspartic acid at the β57 position by a serine, valine, or alanine. This substitution disrupts a salt bridge between β57 aspartic acid and α76 arginine allowing the basic arginine residue to interact with amino acid side chains bound in pocket 9 of I-A^{g7} and DQ8. The present inventors have taken advantage of this unique structural motif, to utilize in silico molecular docking for screening of the National Cancer Institute's (NCI) 140,000 “drug-like” compound library for small molecules capable of binding to pocket 9 of the I-A^{g7} binding groove. Small molecules have been identified can alter specific T cell receptor signaling (TCR) in the presence of their cognate target peptides. With respect to diabetes, the inventors have discovered compounds that enhance insulin peptide presentation to T cells and up regulate IL-10 secretion and prevent the development of diabetes in NOD mice. This discovery provides a new pathway for influencing TCR signaling with small molecules and may relate to the manner in which specific small molecules (drugs and environmental toxins) contribute to immunologic disorders.

The anti-insulin trimolecular complex (MHC-insulin peptide-TCR) was used to study immunologic effects of small organic molecules that can occupy pocket 9 of I-A^{g7}. An insulin B chain peptide, consisting of amino acids 9-23 (B:9-23) is a primary autoantigenic target in the NOD mouse. The majority of CD4⁺ T cell clones within islets of young NOD mice recognize insulin, and more than 90% of such clones target B:9-23. Mutation of this peptide, namely the substitution of alanine for tyrosine at position 16, prevents diabetes in NOD mice. B:9-23 is presented to CD4⁺ TCR by the NOD MHC class II molecule I-A^{g7}.

Recognition of the B9:23 epitope is dependent upon targeting of B:9-23 bound to I-A^{g7} by a conserved “non-stringent” T cell receptor with germline encoded TRAV 5D-04α chain sequence. Of the different variable elements of α/β T cell receptors (Vα, Nα, Jα; Vβ, Nβ, Dβ, Jβ), data indicates that the Vβ TRAV 5D-4*04 sequence (within multiple T cell receptors) is sufficient to engender anti-B:9-23/anti-insulin/anti-islet autoimmunity and diabetes, despite multiple different sequences in the other T cell receptor (TCR) elements. The present inventors have surprisingly found that the Vα TRAV 5D-4*04 sequence (within multiple T cell receptors) is sufficient to engender anti-B:9-23/anti-insulin/anti-islet autoimmunity and diabetes, despite multiple different sequences of the other TCR elements.

The present inventors have demonstrated that therapy with small molecules that alters the trimolecular complex of anti-B:9-23 TRAV5D-4*04 containing T cell receptors/B:12-22/I-A^{g7} prevents diabetes in mouse models, and it is possible to prevent diabetes in NOD mice by targeting TRAV5D-4*04-containing T cell receptors by blocking its target, the B:12-22 peptide in a specific register presented by I-A^{g7}, or by inducing negative selection in the thymus by enhancing TCR reactivity with insulin peptides produced in the thymus. Thus, enhancing binding between the MHC molecule and an insulin peptide will prevent autoimmunity early in the life of a mammal by increasing central tolerance and deletion of T cells that target insulin. Later in the life of the mammal, after the development of the immune system and immune tolerance, the administration of compounds that block or reduce binding between MHC molecule and an insulin peptide may prevent recognition and reduce or eliminate the response of T cells that are already in the periphery.

Because the B:9-23 sequence of insulin 2 is identical in man and mouse and that TCR elements (TRAV5D-04*04 and TRAV 13-1) and MHC (I-A^{g7} and DQA1*0301/DQB1*0302) are highly homologous (and Jα sequences are of secondary importance) these findings can be extended to the treatment and prevention of autoimmune diabetes in humans. In fact, the homologous human elements (DQB1*0302, human insulin B:9-23 peptide, Va TRAV 13-1) also produce a similar trimolecular complex and diabetes risk in “humanized” mice and humans.

Thus, the present invention provides compounds that can modify the binding of T cell receptors to insulin/proinsulin peptides presented by class II MHC molecules, and pharmaceutically acceptable salts and prodrugs thereof. The present invention also provides pharmaceutical compositions containing these compounds. The invention also provides methods of using these compounds and pharmaceutical compositions to prevent or modify the development of autoimmune diseases, including diabetes.

One embodiment of the invention is a method of modifying an autoimmune disease by administering to a mammal in need of such treatment, a therapeutically effective amount of a compound that modifies the T cell response to the targeted antigenic peptide of the autoimmune disease. In a preferred aspect of this embodiment, the compound increases the T cell receptor response to the antigenic peptide. In a more preferred aspect of this embodiment, the compound increases the T cell response to the antigenic peptide by enhancing the binding of the antigenic peptide to a MHC class II molecule that presents the antigenic peptide to a T cell receptor. In a particularly preferred aspect of this embodiment, the compound enhances the binding of an insulin peptide to an MHC class II molecule for presentation to CD4+ T cells, thereby preventing the development of autoimmune diabetes. In another particularly preferred aspect of this embodiment, the compound is at least one of the compounds of the invention that include:

2,4,6-pyridinetricarboxylic acid,

5-formyl-2-hydroxy-2,4-heptadienedioic acid,

1,1,2-cyclopropanetricarboxylic acid,

3-(hydroxy(oxido)amino)-4-phosphonobenzoic acid,

ethane-1,2-diphosphonic acid,

4-oxo-4H-pyran-2,6-dicarboxylic acid,

N,N-bis(phosphonomethyl)glycine,

N-(2-amino-3-carboxypropanoyl)aspartic acid,

1-hydroxy-15-pyridine-2,6-dicarboxylic acid,

2-(acetyloxy)-5-bromobenzoic acid,

3-C-carboxy-2,4-dideoxy-2-ethylpentaric acid,

4-chloro-2-quinolinecarboxylic acid,

3-methyl-1,2-cyclohexanedicarboxylic acid,

(8-quinolinyloxy)acetic acid,

1-propene-1,2,3-tricarboxylic acid,

3-sulfobenzoic acid,

3-(carboxymethyl)benzoic acid,

phenyl dihydrogen phosphate,

2,3-disulfopropanoic acid,

2-phenylethylphosphonic acid,

3-sulfoisonicotinic acid,

2-(phosphonooxy)benzoic acid,

5,6-dichloro-3-hydroxy-2-pyrazinecarboxylic acid,

3-ethyl-4-oxo-1,2-cyclopentanedicarboxylic acid,

N-(aminocarbonyl)aspartic acid,

2-sulfinobenzoic acid,

2,3-dihydroxypropyl dihydrogen phosphate,

4-chlorophthalic acid, phthalic acid,

2-hydroxy-3,5-diiodobenzoic acid,

3,5-dichloro-2-hydroxybenzenesulfinic acid,

4-(hydroxy(oxido)amino)-3-methyl-1H-pyrazole-5-carboxylic acid,

1-amino-1,2,3-propanetricarboxylic acid,

2,6-pyrazinedisulfonic acid,

4-(carboxymethyl)-1H-imidazole-5-carboxylic acid,

2-methyl-1,1,3-propanetricarboxylic acid,

5-chloro-2-hydroxy-3-(hydroxy(oxido)amino)benzoic acid,

1,2-dihydroxy-1,2-ethanedisulfonic acid, and,

2,5-dibromohexanedioic acid.

In another embodiment, the invention is a method of preventing the formation of diabetes in a mammal by administering to the mammal a compound that enhances the T cell response to an insulin peptide presented by an MHC class II molecule. In another preferred aspect of this embodiment, the insulin peptide is B:9-23, the MHC class II molecule is DQ8 and the T cell displays a CD4⁺ TCR. In a preferred aspect of this embodiment, the compound is administered to the mammal in a pharmaceutical composition of the invention. In a related aspect of the invention, the compound is at least one compound of the invention selected from:

N,N-bis(phosphonomethyl) glycine,

Ethane-1,2-diphosphonic acid,

2-methyl-1,1,3-propanetricarboxylic acid,

1,1,2-cyclopropanetricarboxylic acid,

2,3-dihydroxypropyl dihydrogen phosphate,

2-sulfinobenzoic acid, and

1-amino-1,2,3-propanetricarboxylic acid.

In a particularly preferred aspect of the invention, the compound is glysophine (N,N-bis(phosphonomethyl) glycine), or a pharmaceutically acceptable salt thereof.

One embodiment of this invention is a method of preventing or treating autoimmune diseases, or ameliorating the symptoms of these diseases, by administering a therapeutically effective amount of one of these compounds, or a pharmaceutically acceptable salt thereof, or prodrug thereof, to a mammal in need of such treatment or suspected of having an autoimmune disease or having a propensity to develop an autoimmune disease. In a preferred embodiment, the autoimmune disease is autoimmune (Type 1) diabetes.

Another embodiment of this invention is a method of treating an autoimmune disease such as Type 1 diabetes, or ameliorating a symptom thereof, by administering a therapeutically effective combination of at least one of the compounds of the present invention and one or more other known anti-diabetic or anti-inflammatory compounds. For example, other anti-diabetic compounds may include at least one of an alpha-glucosidase inhibitor, a biguanide, a Dpp-4 inhibitor, a meglitinide, a sulfonylurea, a thiazolidinedione or combinations thereof.

Another embodiment of the present invention is a method of modulating the activity of a T cell hybridoma by contacting the cells with at least one compound of the present invention in the presence of class II MHC molecules bound to an insulin protein or to a peptide fragment of an insulin peptide. Preferably, the MHC class H molecule is DQ8 or I-A^{g7}, or a homologous protein, bound to the B:9-23 insulin peptide.

Another embodiment of the present invention is a method of disrupting or otherwise decreasing the binding of a MHC class II molecule bound to an insulin protein or to a peptide fragment of an insulin peptide by contacting the MHC class II molecule with a compound of the present invention in the presence of an insulin protein or to a peptide fragment thereof.

Another embodiment of the present invention is a method of enhancing the binding of an MHC class II molecule bound to an insulin protein, or to a peptide fragment of an insulin peptide, by contacting the MHC class II molecule with a compound of the present invention in the presence of an insulin protein, or to a peptide fragment thereof.

Another embodiment of this invention is a method of testing the susceptibility of a mammal to treatment with one of the compounds of the present invention by testing the mammal for the presence of antibodies to a MHC class II molecule bound to an insulin protein or to a peptide fragment of an insulin peptide, wherein the presence of antibodies that recognize the MHC class II molecules is indicative of the presence or likely development of an autoimmune disease, such as diabetes. In a further embodiment, a mammal found to have antibodies to a MHC class II molecule bound to an insulin protein or to a peptide fragment of an insulin peptide are selected for treatment for diabetes. In a related embodiment, the treatment provided to the mammal selected for treatment includes the administration of at least one therapeutic composition of the present invention.

Additionally, the invention provides pharmaceutical compositions containing one or more of the compounds of the present invention with at least one pharmaceutically acceptable carrier. Also provided herein is a pharmaceutical composition comprising at least one prodrug of the therapeutic compounds of the invention, with at least one pharmaceutically acceptable carrier.

Also provided herein are pharmaceutical packages comprising a pharmaceutical composition comprising therapeutically-effective amounts of at least one therapeutic compound of the invention, optionally together with at least one pharmaceutically acceptable carrier. The pharmaceutical compositions may be administered separately, simultaneously or sequentially, with other compounds or therapies used in the prevention, treatment or amelioration of an autoimmune disease such as diabetes.

Also provided herein are pharmaceutical kits containing a pharmaceutical composition of at least one prodrug of the invention, optionally together with at least one pharmaceutically acceptable carrier; prescribing information and a container. The prescribing information may describe the administration, and/or use of these pharmaceutical compositions alone or in combination with other therapies used in the prevention, treatment or amelioration of an autoimmune disease such as diabetes.

Also provided herein are methods for the prevention, treatment or prophylaxis of diabetes in a mammal comprising administering to a mammal in need thereof therapeutically effective amounts of any of these pharmaceutical compositions, including, for example, the pharmaceutical compositions comprising at least one prodrug of the invention.

Also provided herein are methods for delaying the onset of diabetes in a mammal comprising administering to the mammal therapeutically effective amounts of at least one compound of the invention, including, for example, the pharmaceutical compositions comprising at least one compound of the invention.

Other aspects of the invention will be set forth in the accompanying description of embodiments, which follows and will be apparent from the description or may be learnt by the practice of the invention. However, it should be understood that the following description of embodiments is given by way of illustration only since various changes and modifications within the spirit and scope of the invention will become apparent to those skilled in the art and are encompassed within the scope of this invention.

### BRIEF DESCRIPTION OF DRAWINGS

FIG. 1 shows the results of screening small molecules predicted to bind to pocket 9 of I-Ag7. B:9-23 peptide and small molecule binding to panel a) T cell hybridoma 8-1.1α1, panel b) hybridoma BDC 12-4.1, and panel c) hybridoma BDC 12-4.4. Results are provided as a stimulation index (SI=OD of B:9-23 with compound/OD of tetanus toxin) from triplicate cultures+s.e.m. The data are representative of at least three independent experiments, and 20 of the top 40 scoring compounds are depicted for each T cell hybriodma. Dotted line is at the stimulation index of the B:9-23 peptide. TT=tetanus toxin (negative control peptide). The arrow marks glyphosine, a compound resulting in enhanced TCR stimulation of all three hybridomas.

FIG. 2 shows data demonstrating that glyphosine is specific for insulin B:9-23 presented by I-A^{g7}. The chemical structure of glyphosine (IUPAC chemical nomenclature N,N-Bis(phosphonomethyl)glycine) is shown in panel a. Panel b shows that glyphosine only enhances stimulation of the T cell hybridoma 8-1.1α1 when insulin B:9-23 peptide is present, and with mutated' insulin peptides (shown in panel c). Concentrations are for that of glyphosine and contained B:9-23 peptide unless otherwise noted. The glyphosine concentration used in subsequent experiments was 500 nM. TT=tetanus toxin (negative control peptide). B:19(A)=substitution of the native cysteine for alanine at position 19 in B:9-23. Panel d shows that glyphosine does not alter TCR reactivity to a chromogranin peptide presented by I-A^{g7} to the BDC 2.5 T cell hybridoma. Results are given as a stimulation index (SI) from triplicate cultures+s.e.m. The data are representative of at least three independent experiments. Panel e shows direct binding of biotinylated insulin B:9-23 peptide to I-A^{g7} molecules: increasing concentrations of biotinylated B:9-23 were incubated with I-A^{g7} molecules with and without the addition of 500 nM of glyphosine. The figure is representative of three independent experiments.

FIG. 3 shows that glyphosine stimulates IL-10 production from transgenic, NOD, and in vivo treated NOD splenocytes. Panel a shows individual transgenic BDC 12-4.1 Rag^{-/-} mouse splenocytes cultured without antigen and with B:9-23 peptide in the presence and absence of 500 nM glyphosine. Number of spots for each well is shown in the upper left hand corner. Panel b shows the mean spot number for IL-10 and IFN-γ producing cells from four transgenic mice. Panel c shows IL-10, and panel d shoes IFN-γ, levels measured from the supernatant of the transgenic mice.

FIG. 4 provides additional evidence that glyphosine stimulates IL-10 production from transgenic, NOD, and in vivo treated NOD splenocytes. Panel a shows IL-10 ELISPOT assays from in vitro culture of NOD splenocytes. Each bar represents the mean spot number of triplicates±s.e.m. from 6 mice. Panel b shows IL-10 ELISPOT assays from mice with differing I-A molecules. Each bar represents the mean spot number of triplicates±s.e.m. from at least 4 mice. Panel c shows ex vivo IL-10, and panel d shows IFN-γ, ELISPOT assays from 10 week old female NOD mice treated with 80 mg/kg/day of glyphosine by intraperitoneal administration for 5 days. IL-10 is increased with glyphosine while IFN-γ spot number remains unchanged. Control=in vivo treatment with PBS, Treated=in vivo treatment with glyphosine.

FIG. 5 shows a survival curve of a diabetes prevention study in 4 week old NOD mice. Glyphosine at a dose of 80 mg/kg/day was administered 5 days per week starting at 4 weeks of age until 21 weeks by intraperitoneal administration. The glyphosine treated mice are statistically different (p<0.001) compared to controls receiving phosphate buffered saline.

### DESCRIPTION OF EMBODIMENTS

The present inventors have discovered that small organic molecules identified to bind pocket 9 of I-A^{g7} enhance T cell receptor (TCR) responses to the B:9-23 insulin peptide both in vitro and in vivo, and dramatically upregulate IL-10 production. While compounds that would inhibit binding of the B:9-23 peptide to I-A^{g7} would intuitively be expected to prevent or treat autoimmune diseases, and particularly diabetes, these surprising results reveal an immunomodulatory action of these small organic molecules that enhance the TCR responses, potentially through the up regulation of IL-10.

The insulin B:9-23 peptide can bind to I-A^{g7} in multiple different registers, and in particular the BDC 12-4.1 T cell receptor recognizes the B:12-23 peptide in a low affinity register. By apparently changing the charge of pocket 9 of the MHC class II molecule, these small organic molecules favor peptide binding in this low affinity register as well as alter the TCR interaction with I-A^{g7}-bound insulin peptides. This remarkable IL-10 response may reflect this altered TCR interaction. IL-10 is associated with Tr1 regulatory T cells and may also up regulate additional regulatory T cell responses. These small organic molecules effectively delay diabetes development in mammals, and enhanced IL-10 production following the treatment may contribute to this protection from diabetes.

Thus, the present invention is drawn to methods of preventing or treating autoimmune diseases such as diabetes by modulating the binding of MHC class II molecules to antigenic peptides or fragments of antigenic peptides of the autoimmune disease by the administration of compounds of the invention, or pharmaceutically-acceptable salts and/or prodrugs thereof to a mammal.

The term “insulin peptide” is used to denote a peptide fragment of an insulin protein. Although the fragment is typically a subset of the amino acid sequence of the insulin protein, an insulin peptide may contain the entire amino acid sequence of a naturally-occurring insulin protein.

“Modulate” means to alter the ability of an antigenic peptide to associate with an MHC protein molecule, for example, an insulin peptide associated with autoimmune diabetes to associate with a MHC class II protein molecule. Thus, modulation includes enhancement of the association between an insulin peptide and a MHC class II protein molecule, as well as dissociation of a bound complex formed by the association of an insulin peptide bound to a MHC class II protein molecule as well as preventing the formation of a complex between an insulin peptide and a MHC class II protein molecule.

As used herein, the term “agent” means a chemical or biological molecule such as a simple or complex organic molecule, a peptide, a protein or an oligonucleotide.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

“Pharmaceutically-acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines, or alkali or organic salts of acidic residues such as carboxylic acids. Pharmaceutically-acceptable salts include the conventional non-toxic salts or the quaternary ammonium salts of the parent compound formed, for example, from non-toxic inorganic or organic acids. Such conventional nontoxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Pharmaceutically acceptable salts are those forms of compounds, suitable for use in contact with the tissues of human beings and animals without excessive toxicity, irritation, allergic response, or other problem or complication, commensurate with a reasonable benefit/risk ratio.

Pharmaceutically-acceptable salt forms of compounds provided herein are synthesized from the parent compound which contains a basic or acidic moiety by conventional chemical methods. Generally, such salts are, for example, prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in at page 1418 of Remington's Pharmaceutical Sciences, 17th ed., Mack Publishing Company, Easton, Pa., 1985.

“Prodrugs” are intended to include any covalently bonded carriers that release an active parent drug of the present invention in vivo when such prodrug is administered to a mammalian subject. Since prodrugs are known to enhance numerous desirable qualities of pharmaceuticals (i.e., solubility, bioavailability, half life, manufacturing, etc.) the compounds of the present invention may be delivered in prodrug form. Thus, the present invention is intended to cover prodrugs of the presently claimed compounds, methods of delivering the same, and compositions containing the same. Prodrugs of the present invention are prepared by modifying functional groups present in the compound in such a way that the modifications are cleaved, either in routine manipulation or in vivo, to a compound of the invention. Prodrugs include compounds of the present invention wherein an acyl, hydroxy, amino, or sulfhydryl group is bonded to any group that, when the prodrug of the present invention is administered to a mammalian subject, is cleaved to form a free acetyl, hydroxyl, free amino, or free sulfydryl group, respectively. Examples of prodrugs include, but are not limited to, acetate, formate, and benzoate derivatives of alcohol and amine functional groups in the compounds of the present invention.

The term “therapeutically-effective amount” of a compound of this invention means an amount effective to modulate the formation or progression of an autoimmune disorder in a host.

It will be appreciated by those skilled in the art that compounds of the invention having a chiral center may exist in, and may be isolated in, optically active and racemic forms. It is to be understood that the compounds of the present invention encompasses any racemic, optically-active, regioisomeric or stereoisomeric form, or mixtures thereof, which possess the therapeutically useful properties described herein. It is well known in the art how to prepare optically active forms (for example, by resolution of the racemic form by recrystallization techniques, by synthesis from optically-active starting materials, by chiral synthesis, or by chromatographic separation using a chiral stationary phase). It is also to be understood that the scope of this invention encompasses not only the various isomers, which may exist but also the various mixtures of isomers, which may be formed. For example, if the compound of the present invention contains one or more chiral centers, the compound can be synthesized enantioselectively or a mixture of enantiomers and/or diastereomers can be prepared and separated. The resolution of the compounds of the present invention, their starting materials and/or the intermediates may be carried out by known procedures, e.g., as described in the four volume compendium Optical Resolution Procedures for Chemical Compounds: Optical Resolution Information Center, Manhattan College, Riverdale, N.Y., and in Enantiomers, Racemates and Resolutions, Jean Jacques, Andre Collet and Samuel H. Wilen; John Wiley & Sons, Inc., New York, 1981, which is incorporated in its entirety by this reference. Basically, the resolution of the compounds is based on the differences in the physical properties of diastereomers by attachment, either chemically or enzymatically, of an enantiomerically pure moiety resulting in forms that are separable by fractional crystallization, distillation or chromatography.

The compounds used in making the pharmaceutical compositions of the present invention may be purchased commercially. The compounds of the present invention, including the salts and prodrugs of these compounds, may also be prepared in ways well known to one skilled in the art of organic synthesis. These compounds of this invention may be prepared using the reactions performed in solvents appropriate to the reagents and materials employed and suitable for the transformation being effected. It is understood by one skilled in the art of organic synthesis that the functionality present on various portions of the molecule must be compatible with the reagents and reactions proposed. Such restrictions to the substituents, which are compatible with the reaction conditions, will be readily apparent to one skilled in the art and alternate methods must then be used.

Also provided herein are pharmaceutical compositions containing compounds of the invention and a pharmaceutically-acceptable carrier, which are media generally accepted in the art for the delivery of biologically active agents to animals, in particular, mammals. Pharmaceutically-acceptable' carriers are formulated according to a number of factors well within the purview of those of ordinary skill in the art to determine and accommodate. These include, without limitation: the type and nature of the active agent being formulated; the subject to which the agent-containing composition is to be administered; the intended route of administration of the composition; and, the therapeutic indication being targeted. Pharmaceutically-acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, well known to those of ordinary skill in the art. Descriptions of suitable pharmaceutically-acceptable carriers, and factors involved in their selection, are found in a variety of readily available sources, such as Remington's Pharmaceutical Sciences, 17th ed., Mack Publishing Company, Easton, Pa., 1985.

This invention further provides a method of treating a mammal afflicted with an autoimmune disorder or preventing a mammal from developing autoimmunity, which includes administering to the mammal a pharmaceutical composition provided herein. Such compositions generally comprise a therapeutically effective amount of a compound provided herein, that is, an amount effective to prevent, ameliorate, lessen or inhibit an autoimmune disease. Such amounts typically comprise from about 0.1 to about 1000 mg of the compound per kilogram of body weight of the mammal to which the composition is administered. Therapeutically effective amounts can be administered according to any dosing regimen satisfactory to those of ordinary skill in the art.

Administration may be, for example, by various parenteral means. Pharmaceutical compositions suitable for parenteral administration include various aqueous media such as aqueous dextrose and saline solutions; glycol solutions are also useful carriers, and preferably contain a water soluble salt of the active ingredient, suitable stabilizing agents, and if necessary, buffering agents. Antioxidizing agents, such as sodium bisulfite, sodium sulfite, or ascorbic acid, either alone or in combination, are suitable stabilizing agents; also used are citric acid and its salts, and EDTA. In addition, parenteral solutions can contain preservatives such as benzalkonium chloride, methyl- or propyl-paraben, and chlorobutanol.

Alternatively, compositions can be administered orally in solid dosage forms, such as capsules, tablets and powders; or in liquid forms such as elixirs, syrups, and/or suspensions. Gelatin capsules can be used to contain the active ingredient and a suitable carrier such as, but not limited to, lactose, starch, magnesium stearate, stearic acid, or cellulose derivatives. Similar diluents can be used to make compressed tablets. Both tablets and capsules can be manufactured as sustained release products to provide for continuous release of medication over a period of time. Compressed tablets can be sugar-coated or film-coated to mask any unpleasant taste, or used to protect the active ingredients from the atmosphere, or to allow selective disintegration of the tablet in the gastrointestinal tract.

A preferred formulation of the invention is a mono-phasic pharmaceutical composition suitable for parenteral or oral administration for the prevention, treatment or prophylaxis of an autoimmune disease such as diabetes, consisting essentially of a therapeutically-effective amount of a compound of the invention, and a pharmaceutically acceptable carrier.

Another preferred formulation of the invention is a mono-phasic pharmaceutical composition suitable for parenteral or oral administration for the prevention, treatment or prophylaxis of an autoimmune disease such as diabetes, consisting essentially of a therapeutically-effective amount of a prodrug of the therapeutic compounds of the invention, and a pharmaceutically acceptable carrier.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as wetting agents, emulsifying agents and dispersing agents. It may also be desirable to include isotonic agents, such as sugars, sodium chloride, and the like in the compositions. In addition, prolonged absorption of the injectable pharmaceutical form may be brought about by the inclusion of agents which delay absorption such as aluminum monosterate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn may depend upon crystal size and crystalline form. Alternatively, delayed absorption of a parenterally-administered drug is accomplished by dissolving or suspending the drug in an oil vehicle.

Injectable depot forms are made by forming microencapsule matrices of the drug in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue. The injectable materials can be sterilized for example, by filtration through a bacterial-retaining filter.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the therapeutic compounds of the present invention.

Formulations of the invention suitable for oral administration may be in the form of capsules, cachets, pills, tablets, powders, granules or as a solution or a suspension in an aqueous or non-aqueous liquid, or an oil-in-water or water-in-oil liquid emulsions, or as an elixir or syrup, or as pastilles (using an inert base, such as gelatin and glycerin, or sucrose and acacia), and the like, each containing a predetermined amount of a compound or compounds of the present invention as an active ingredient. A compound or compounds of the present invention may also be administered as bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monosterate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may be employed as fillers in soft and hard-filled gelatin capsules using such excipients as lactose or milk sugars, as well as high molecular weight polyethylene glycols and the like.

A tablet may be made by compression or molding optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter. These compositions may also optionally contain opacifying agents and may be of a composition that they release the active ingredient only, or preferentially, in a certain portion of the gastrointestinal tract, optionally, in a delayed manner. Examples of embedding compositions which can be used include polymeric substances and waxes. The active ingredient can also be in microencapsulated form.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Liquid dosage forms for oral administration of the compounds of the invention include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or salicylate, and which is solid at room temperature, but liquid at body temperature and, therefore, will melt in the rectum or vaginal cavity and release the active compound. Formulations of the present invention which are suitable for vaginal administration also include pessaries, tampons, creams, gels, pastes, foams or spray formulations containing such carriers as are known in the art to be appropriate.

Dosage forms for the topical or transdermal administration of compounds of this invention include powders, sprays, ointments, pastes, creams, lotions, gels, solutions, patches, drops and inhalants. The active ingredient may be mixed under sterile conditions with a pharmaceutically-acceptable carrier, and with any buffers, or propellants which may be required.

The ointments, pastes, creams and gels may contain, in addition to an active ingredient, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active ingredient, excipients such as lactose, talc, silicic acid, aluminum hydroxide, calcium silicates and polyamide powder or mixtures of these substances. Sprays can additionally contain customary propellants such as chlorofluorohydrocarbons and volatile unsubstituted hydrocarbons, such as butane and propane.

Transdermal patches have the added advantage of providing controlled delivery of compounds of the invention to the body. Such dosage forms can be made by dissolving, dispersing or otherwise incorporating one or more compounds of the invention in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

Pharmaceutical formulations include those suitable for administration by inhalation or insufflation or for nasal or intraocular administration. For administration to the upper (nasal) or lower respiratory tract by inhalation, the compounds of the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of one or more compounds of the invention and a suitable powder base, such as lactose or starch. The powder composition may be presented in unit dosage form in, for example, capsules or cartridges, or, e.g., gelatin or blister packs from which the powder may be administered with the aid of an inhalator, insufflator or a metered-dose inhaler.

For intranasal administration, compounds of the invention may be administered by means of nose drops or a liquid spray, such as by means of a plastic bottle atomizer or metered-dose inhaler. Typical of atomizers are the Mistometer (Wintrop) and Medihaler (Riker).

Drops, such as eye drops or nose drops, may be formulated with an aqueous or nonaqueous base also comprising one or more dispersing agents, solubilizing agents or suspending agents. Liquid sprays are conveniently delivered from pressurized packs. Drops can be delivered by means of a simple eye dropper-capped bottle or by means of a plastic bottle adapted to deliver liquid contents dropwise by means of a specially shaped closure.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

The dosage formulations provided by this invention may contain the therapeutic compounds of the invention, either alone or in combination with other therapeutically active ingredients, and pharmaceutically acceptable inert excipients. The term ‘pharmaceutically acceptable inert excipients’ includes at least one of diluents, binders, lubricants/glidants, coloring agents and release modifying polymers.

Suitable antioxidants may be selected from amongst one or more pharmaceutically acceptable antioxidants known in the art. Examples of pharmaceutically acceptable antioxidants include butylated hydroxyanisole (BHA), sodium ascorbate, butylated hydroxytoluene (BHT), sodium sulfite, citric acid, malic acid and ascorbic acid. The antioxidants may be present in the dosage formulations of the present invention at a concentration between about 0.001% to about 5%, by weight, of the dosage formulation.

Suitable chelating agents may be selected from amongst one or more chelating agents known in the art. Examples of suitable chelating agents include disodium edetate (EDTA), edetic acid, citric acid and combinations thereof. The chelating agents may be present in a concentration between about 0.001% and about 5%, by weight, of the dosage formulation.

The dosage form may include one or more diluents such as lactose, sugar, cornstarch, modified cornstarch, mannitol, sorbitol, and/or cellulose derivatives such as wood cellulose and microcrystalline cellulose, typically in an amount within the range of from about 20% to about 80%, by weight.

The dosage form may include one or more binders in an amount of up to about 60% w/w. Examples of suitable binders include methyl cellulose, hydroxypropyl cellulose, hydroxypropylmethyl cellulose, polyvinyl pyrrolidone, eudragits, ethyl cellulose, gelatin, gum arabic, polyvinyl alcohol, pullulan, carbomer, pregelatinized starch, agar, tragacanth, sodium alginate, microcrystalline cellulose and the like.

Examples of suitable disintegrants include sodium starch glycolate, croscarmellose sodium, crospovidone, low substituted hydroxypropyl cellulose, and the like. The concentration may vary from 0.1% to 15%, by weight, of the dosage form.

Examples of lubricants/glidants include colloidal silicon dioxide, stearic acid, magnesium stearate, calcium stearate, talc, hydrogenated castor oil, sucrose esters of fatty acid, microcrystalline wax, yellow beeswax, white beeswax, and the like. The concentration may vary from 0.1% to 15%, by weight, of the dosage form.

Release modifying polymers may be used to form extended release formulations containing the therapeutic compounds of the invention. The release modifying polymers may be either water-soluble polymers, or water insoluble polymers. Examples of water-soluble polymers include polyvinylpyrrolidone, hydroxy propylcellulose, hydroxypropyl methylcellulose, vinyl acetate copolymers, polyethylene oxide, polysaccharides (such as alginate, xanthan gum, etc.), methylcellulose and mixtures thereof. Examples of water-insoluble polymers include acrylates such as methacrylates, acrylic acid copolymers; cellulose derivatives such as ethylcellulose or cellulose acetate; polyethylene, and high molecular weight polyvinyl alcohols.

Another embodiment of the invention relates to the use of any of the prodrug compounds or compositions described herein in the preparation of a medicament for the treatment of an autoimmune disease such as diabetes.

Also encompassed by the present invention are methods for screening potential therapeutic agents that modulate the interaction between class II MHC molecules and insulin peptides comprising the steps of: (a) combining an MHC protein molecule and an insulin peptide under conditions in which they interact, in the presence of a potential therapeutic agent, and; (b) monitoring the interaction of the MHC molecule and insulin peptide; wherein a potential therapeutic agent is selected for further study when it modifies the interaction compared to a control sample to which no potential therapeutic agent has been added. In one embodiment, the potential therapeutic agent is selected from the group consisting of a pharmaceutical agent, a cytokine, a small molecule drug, a cell-permeable small molecule drug, a hormone, a combination of interleukins, a lectin, a bispecific antibody, a peptide mimetic, and a sense or antisense oligonucleotide. In another embodiment, the MHC molecule is a class II MHC molecule. In a preferred embodiment, the class II MHC molecule is I-A^{g7} or DQ8 that has a similar pocket 9 binding region lacking an aspartic acid at position 57 of the beta chain and associated with diabetes risk in man (DQ8) or mouse (IA-g7) or a fragment of I-A^{g7} sufficient to effect binding to an insulin peptide, or a fusion protein comprising a portion of I-A^{g7} sufficient to effect binding to an insulin peptide. The fusion protein may comprise a labeled I-A^{g7}.

The screening assay can be performed by allowing the class II MHC molecule to interact with an insulin peptide, then adding a potential therapeutic agent to be tested. Control reactions will not contain the agent. Following incubation of the reaction mixture under conditions known to be favorable for the association of the MHC molecule and insulin peptide in the absence of a test agent, the amount of insulin peptide specifically bound to the MHC molecule in the presence of a test agent can be determined. For ease of detecting binding, the insulin protein can be labeled with a detectable moiety, such as a radionuclide or a fluorescent label, using methods well known in the art. By comparing the amount of specific binding of the insulin peptide and the MHC molecule in the presence of a test agent, as compared to the control level of binding, an agent that increases or decreases the binding of insulin peptides and class II MHC molecules can be identified. Thus, this drug screening assay provides a rapid and simple method for selecting drugs having a desirable effect on the association of an insulin peptide and a MHC molecule.

In one embodiment of the present invention, the monitoring step includes exposure of the I-A^{g7} to a T cell to evaluate the response of the T cell. The T cell may be an in vitro T cell hybridoma population, such as, but not limited to, BDC 12-4.1 and/or BDC 2.5 T cell hybridomas.

In one embodiment, the monitoring of the T cell response reveals a proliferation of T cells, or increased T cell activity, following contact with the class II MHC molecule compared to the T cell response seen following contact with the class II MHC molecule in the absence of the potential therapeutic agent, and the potential therapeutic agent is designated as enhancing the interaction between class II MHC molecules and insulin peptides. While such enhancing agent may have been expected to stimulate or enhance the development or progression of an autoimmune disease, the present inventors have discovered that compounds that enhance the interaction between class II MHC molecules and insulin peptides can prevent the formation of and ameliorate the signs of diabetes.

In another embodiment, this monitoring of the T cell response reveals a decrease in T cell number or activity following contact with the class II MHC molecule compared to the T cell response seen following contact with the class II MHC molecule in the absence of the potential therapeutic agent, and the potential therapeutic agent is designated as inhibiting the interaction between class II MHC molecules and insulin peptides. Such inhibiting agents can inhibit or decrease the progression of an autoimmune disease such as diabetes by enhancing central deletion or altering T cell receptor signaling.

In one embodiment, the drug screening assay can utilize a MHC molecule fusion protein such as a MHC molecule-insulin peptide fusion protein. The fusion protein is characterized, in part, by eliciting a response from a T cell. Where such a fusion protein is used in the assay, the potential therapeutic agent is selected for its effect on the response from the T cell population, wherein the potential therapeutic agent may enhance or inhibit the response from the T cell population to the fusion protein based on effects imparted by the agent on the fusion protein or the interaction between the fusion protein and the T cells.

Another embodiment of the invention relates to the use of any of the compounds or compositions described herein in the preparation of a medicament for the modulation of an autoimmune disease. The modulation may include the prevention or treatment of an autoimmune disease, such as diabetes, in a mammal.

Each publication or patent cited herein is incorporated herein by reference in its entirety.

Additional objects, advantages, and novel features of this invention will become apparent to those skilled in the art upon examination of the following examples thereof, which are not intended to be limiting.

### EXAMPLES

### Example 1

### Selection of Drug Candidates

To identify candidate molecules predicted to “dock” in pocket 9 of the I-A^{g7} binding groove, a supercomputer was used to screen 140,000 small molecules from the NCI library of “drug-like” compounds (NCI Developmental Therapeutics Program's (NCI/DTP) repository). Existing crystal structures available for modeling include I-A^{g7} with a bound GAD65 peptide and B:9-23 bound to DQ8, but not I-A^{g7} with the B:9-23 peptide. The antigen binding clefts of DQ8 and I-A^{g7} were superimposed to examine the critical contacts. The B:9-23 peptide was displayed with I-A^{g7} in the orientation and conformation bound to DQ8. This conformation of the insulin B:9-23 peptide was complementary with the antigen binding cleft of the crystal structure of I-A^{g7}. All NCI organic compounds were docked in 1000 orientations using the DOCK v5.1.0 program algorithm {111} and scored based on a combination of polar and non-polar interactions.

### Example 2

### In Vitro Testing of Compounds

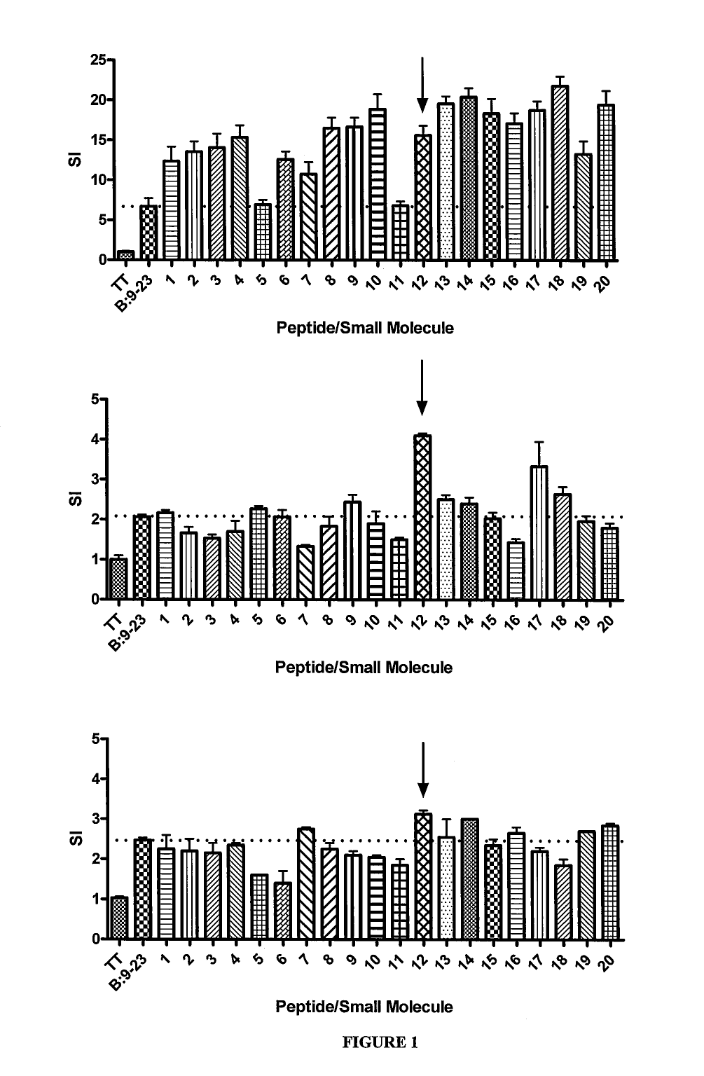

The top 40 scoring compounds (shown in Table 1) were screened for their ability to alter anti-B:9-23 T cell responses to three different T cell hybridomas, all with the dominant conserved Vα5D-4 TCR element but different CDR3α, Jα, and TCRβ chains. FIG. 1 shows the results of this in vitro testing. Multiple compounds enhanced TCR signaling of the 8-1.1α1 hybridoma, while fewer compounds stimulated the BDC 12-4.1 and 12-4.4 hybridomas. Given the positively charged arginine in pocket 9 of I-A^{g7}, each of the top 40 compounds are negatively charged. The same assays evaluating hybridoma response were performed using 40 random compounds from the NCI/DTP repository. Testing of these control compounds did not result in stimulation above insulin B:9-23 peptide alone with any of the three hybridomas.

TABLE 1
Top 40 scoring small molecules for pocket 9 of I-A^{g7}

| Compound Name/ | | | | | |
|---|---|---|---|---|---|
| Chemical Formula/ | | | | | |
| Molecular Weight/ | | Docking | Hybridoma Response | | |
| NCI Number | Structure | Score | 8-1.1α₁ | 12-4.1 | 12-4.4 |
| 2,4,6-pyridinetricarboxylic acid (ACD/Name 4.0) C8H5NO6 211.1306 403251 | 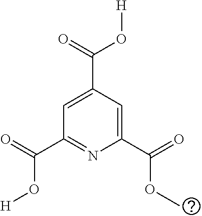 | −36.9 | + | − | − |
| 5-formyl-2-hydroxy-2,4- heptadienedioic acid (ACD/Name 4.0) C8H8O6 200.1476 318505 | 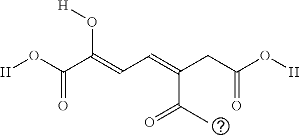 | −32.6 | + | − | − |
| 1,1,2-cyclopropanetricarboxylic acid (ACD/Name 4.0) C6H6O6 174.1098 24681 | 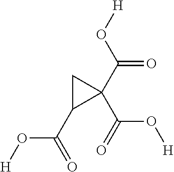 | −32.2 | + | − | − |
| 3-(hydroxy(oxido)amino)-4- phosphonobenzoic acid (ACD/Name 4.0) C7H6NO7P 247.1007 129464 | 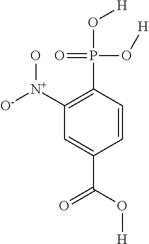 | −32.2 | − | − | − |
| Ethane-1,2-diphosphonic acid C2H8O6P2 190.0291 40837 | 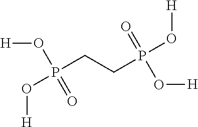 | −32.0 | + | + | − |
| 4-oxo-4H-pyran-2,6-dicarboxylic acid (ACD/Name 4.0) C7H4O6 184.105 3979 | 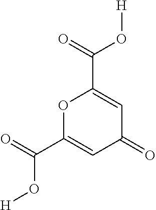 | −31.6 | + | − | − |
| Glyphosine N,N-bis(phosphonomethyl)glycine (ACD/Name 4.0) C4H11NO8P2 263.0803 18468 | 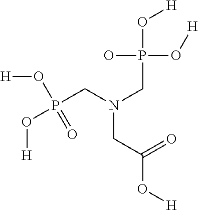 | −31.1 | + | + | + |
| N-(2-amino-3- carboxypropanoyl)aspartic acid (ACD/Name 4.0) C8H12N2O7 248.192 332639 | 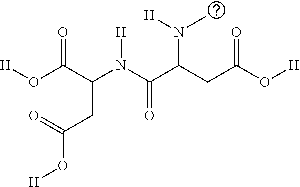 | −31.0 | − | − | − |
| (No Name) C4H12O6P2 218.0827 407819 | 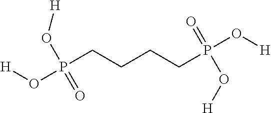 | −30.9 | + | − | − |
| 1-hydroxy-15-pyridine-2,6- dicarboxylic acid (ACD/Name 4.0) C7H5NO5 183.1202 78435 | 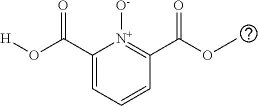 | −30.9 | − | − | − |
| 2-(acetyloxy)-5-bromobenzoic acid (ACD/Name 4.0) C9H7BrO4 259.0559 2400 | 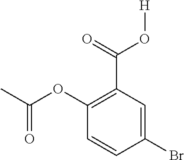 | −30.6 | + | − | − |
| 3-C-carboxy-2,4-dideoxy-2- ethylpentaric acid (ACD/Name 4.0) C8H12O7 220.1786 141845 | 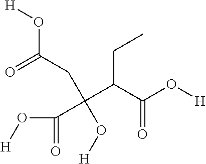 | −30.5 | + | − | − |
| 4-chloro-2-quinolinecarboxylic acid (ACD/Name 4.0) C10H6ClNO2 207.6159 136919 | 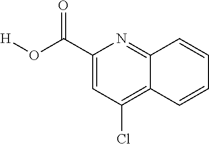 | −30.4 | − | − | − |
| 3-methyl-1,2- cyclohexanedicarboxylic acid (ACD/Name 4.0) C9H14O4 186.2072 30861 | 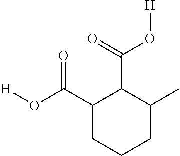 | −30.3 | + | − | − |
| (8-quinolinyloxy)acetic acid (ACD/Name 4.0) C11H9NO3 203.197 4082 | 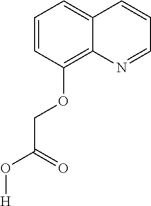 | −30.1 | − | − | − |
| 1-propene-1,2,3-tricarboxylic acid (ACD/Name 4.0) C6H6O6 174.1098 43980 | 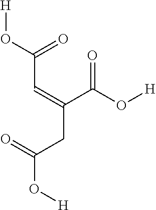 | −30.0 | − | − | − |
| 3-sulfobenzoic acid (ACD/Name 4.0) C7H6O5S 202.1814 2625 | 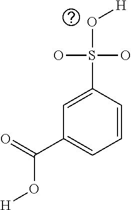 | −30.0 | + | − | − |
| 3-(carboxymethyl)benzoic acid (ACD/Name 4.0) C9H8O4 180.1598 108368 | 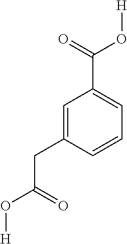 | −30.0 | − | − | − |
| phenyl dihydrogen phosphate (ACD/Name 4.0) C6H7O4P 174.0927 7190 | 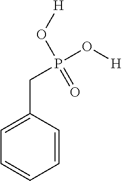 | −29.9 | + | − | − |
| 2,3-disulfopropanoic acid (ACD/Name 4.0) C3H6O8S2 234.1956 229631 | 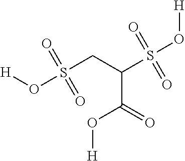 | −29.9 | − | − | − |
| 2-phenylethylphosphonic acid (ACD/Name 4.0) C8H11O3P 186.1469 140288 | 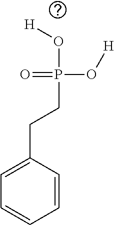 | −29.9 | − | − | − |
| 3-sulfoisonicotinic acid (ACD/Name 4.0) C6H5NO5S 203.1692 74449 | 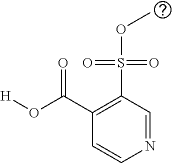 | −29.9 | − | − | − |
| 2-(phosphonooxy)benzoic acid (ACD/Name 4.0) C7H7O6P 218.1025 46475 | 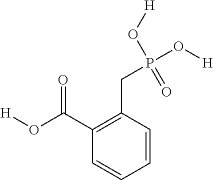 | −29.7 | − | − | − |
| 5,6-dichloro-3-hydroxy-2- pyrazinecarboxylic acid (ACD/Name 4.0) C5H2Cl2N2O3 208.9884 382682 | 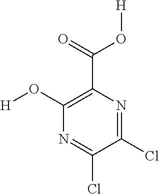 | −29.7 | − | − | − |
| 3-ethyl-4-oxo-1,2- cyclopentanedicarboxylic acid (ACD/Name 4.0) C9H12O5 200.1908 215985 | 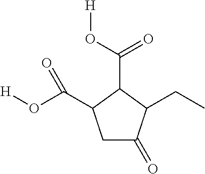 | −29.6 | − | − | − |
| N-(aminocarbonyl)aspartic acid (ACD/Name 4.0) C5H8N2O5 176.1286 14983 | 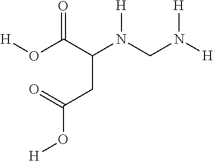 | −29.6 | + | − | − |
| 2-sulfinobenzoic acid (ACD/Name 4.0) C7H6O4S 186.182 179320 | 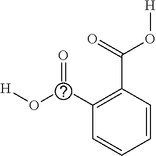 | −29.6 | − | − | − |
| 2,3-dihydroxypropyl dihydrogen phosphate (ACD/Name 4.0) C3H9O6P 172.0743 9231 | 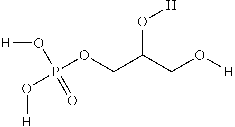 | −29.5 | + | − | − |
| 4-chlorophthalic acid (ACD/Name 4.0) C8H5ClO4 200.5781 57755 | 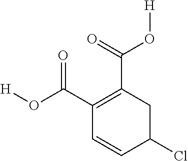 | −29.5 | − | − | − |
| phthalic acid (ACD/Name 4.0) C8H6O4 166.133 5348 | 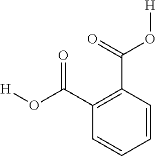 | −29.5 | − | − | − |
| 2-hydroxy-3,5-diiodobenzoic acid (ACD/Name 4.0) C7H4I2O3 389.9158 6303 | 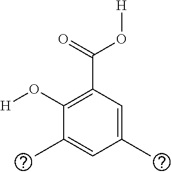 | −29.5 | − | − | − |
| 3,5-dichloro-2- hydroxybenzenesulfinic acid (ACD/Name 4.0) C6H4Cl2O3S 227.0618 624184 | 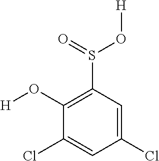 | −29.5 | + | − | − |
| 4-(hydroxy(oxido)amino)-3-methyl- 1H-pyrazole-5-carboxylic acid (ACD/Name 4.0) C5H5N3O4 171.1122 1409 | 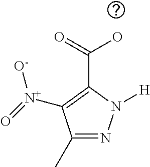 | −29.4 | + | − | − |
| 1-amino-1,2,3- propanetricarboxylic acid (ACD/Name 4.0) C6H9NO6 191.1402 206248 | 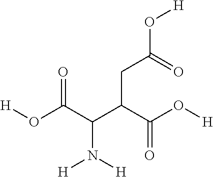 | −29.4 | + | − | − |
| 2,6-pyrazinedisulfonic acid (ACD/Name 4.0) C4H4N2O6S2 240.2054 156622 | 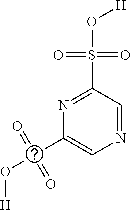 | −29.4 | − | − | − |
| 4-(carboxymethyl)-1H-imidazole- 5-carboxylic acid (ACD/Name 4.0) C6H6N2O4 170.1244 40384 | 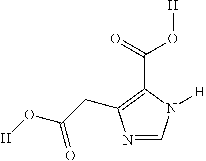 | −29.2 | + | + | − |
| 2-methyl-1,1,3- propanetricarboxylic acid (ACD/Name 4.0) C7H10O6 190.1524 25950 | 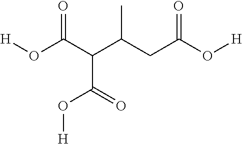 | −29.2 | + | − | − |
| 5-chloro-2-hydroxy-3- (hydroxy(oxido)amino)benzoic acid (ACD/Name 4.0) C7H4ClNO5 217.5653 37272 | 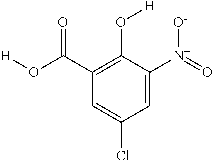 | −29.2 | + | − | − |
| 1,2-dihydroxy-1,2- ethanedisulfonic acid (ACD/Name 4.0) C2H6O8S2 222.1846 18262 | 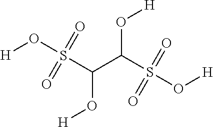 | −29.1 | − | − | − |
| 2,5-dibromohexanedioic acid (ACD/Name 4.0) C6H8Br2O4 303.9348 243606 | 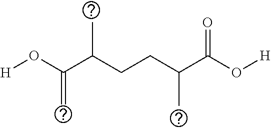 | −28.9 | − | − | − |

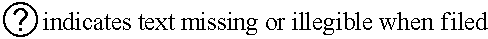

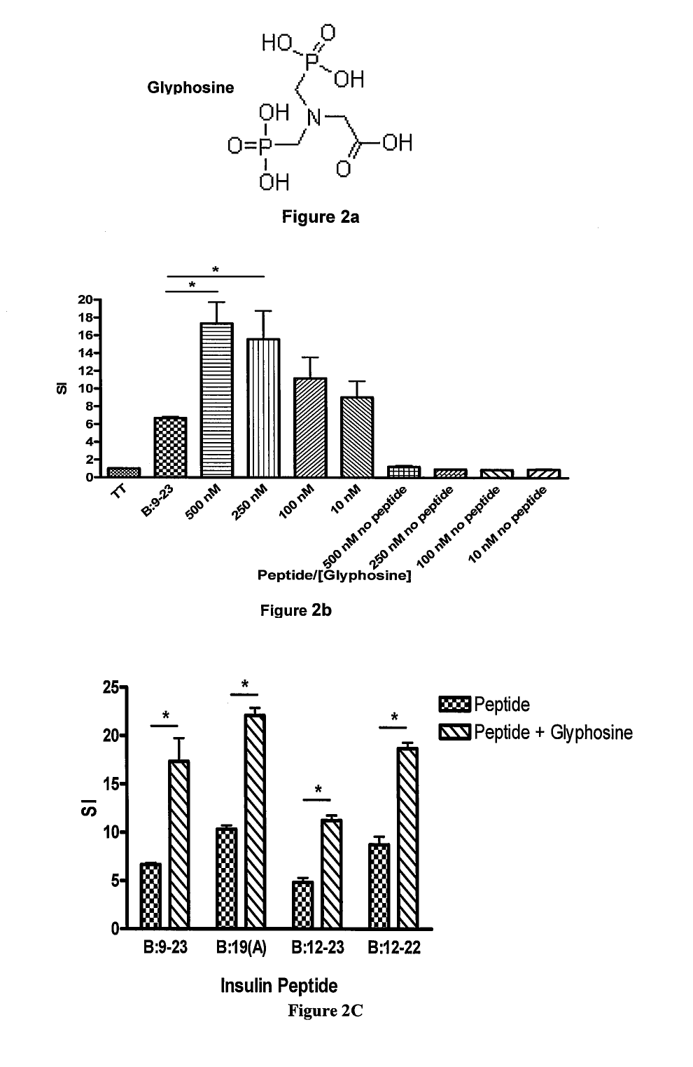

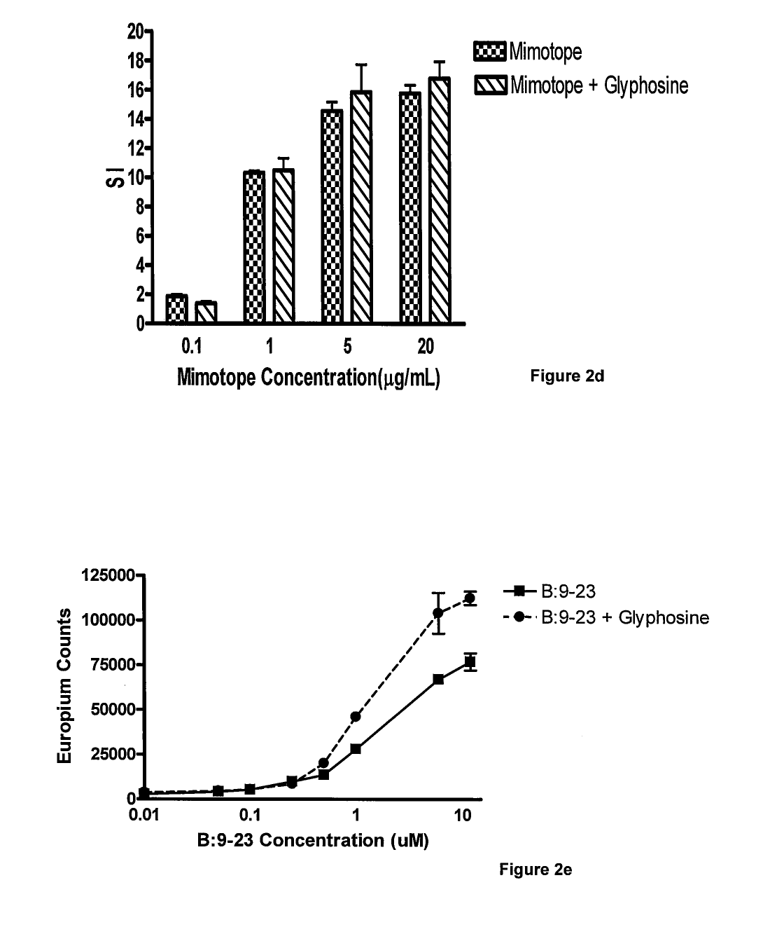

In the initial screening of T cell hybridomas one compound glyphosine, depicted in FIG. 2, enhanced TCR stimulation with B:9-23 when tested with all three anti-B:9-23 hybridomas. Glyphosine is a plant growth inhibitor which increases sucrose content and has previously been used to ripen sugarcane prior to harvesting. Glyphosine inhibits the enzyme phosphoenolpyruvate carboxylase, not required in mammals but required in plants for photosynthesis. Glyphosine modulated TCR stimulation only when peptide is present (FIG. 2b). An effect was seen down to a concentration of 10 nM with an EC50 concentration of 70.6 nM. The maximal stimulatory effect was at a concentration of 500 nM, approximately 2.5 fold greater than peptide alone. Glyphosine also enhanced TCR stimulation in the presence of other modified B:9-23 insulin peptides (FIG. 2c). To test whether glyphosine enhances TCR reactivity specifically to the B:9-23 peptide, a chromogranin peptide mimotope presented by I-A^{g7} to the BDC 2.5 T cell hybridoma was studied. Glyphosine did not enhance peptide stimulation of this hybridoma (FIG. 2d).

### Example 3

### Effects of Glyphosine on Peptide Binding to Purified I-A^{g7}

To document direct effects of glyphosine on peptide binding to purified I-A^{g7} an I-A^{g7} protein construct was expressed in baculovirus with linked peptide. The flexible linker contained a thrombin cleavage site, allowing for thrombin cleavage of the linker and release of the peptide. Glyphosine was able to directly enhance B:9-23 peptide binding to the empty I-A^{g7} over a wide concentration range (FIG. 2, panel e). The binding curve shown in panel e of FIG. 2 did not change based upon time of incubation, 2 hours versus 24 hours, suggesting that glyphosine changes the equilibrium of the B:9-23/MHC class II binding reaction and does not simply catalyze B:9-23 peptide binding to I-A^{g7}.

### Example 4

### In Vitro Response of a Monoclonal T Cell Population Targeted to B:9-23 Peptide

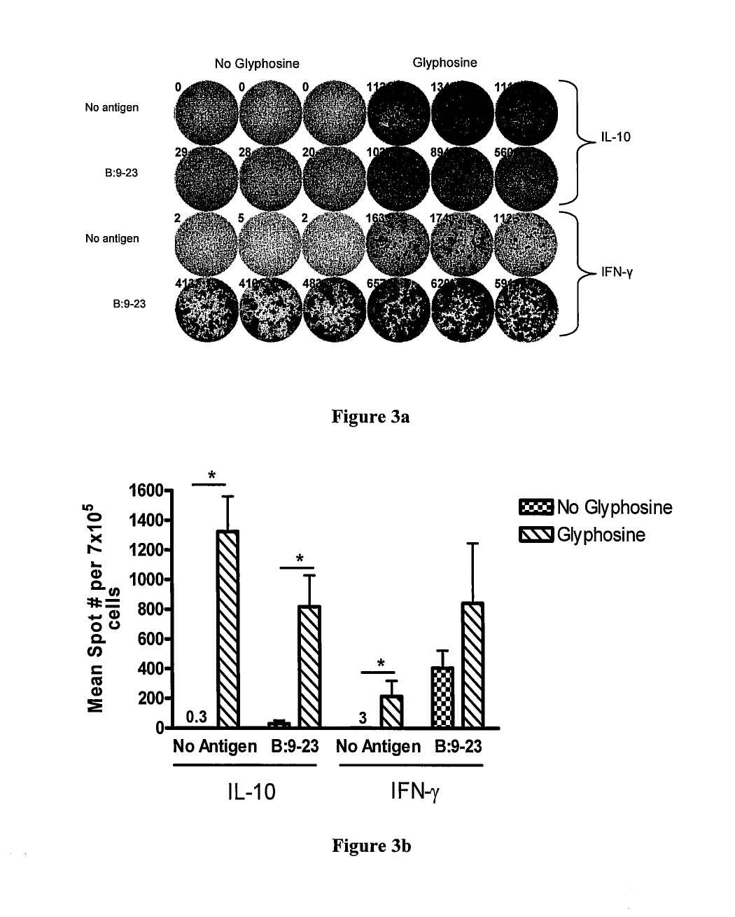

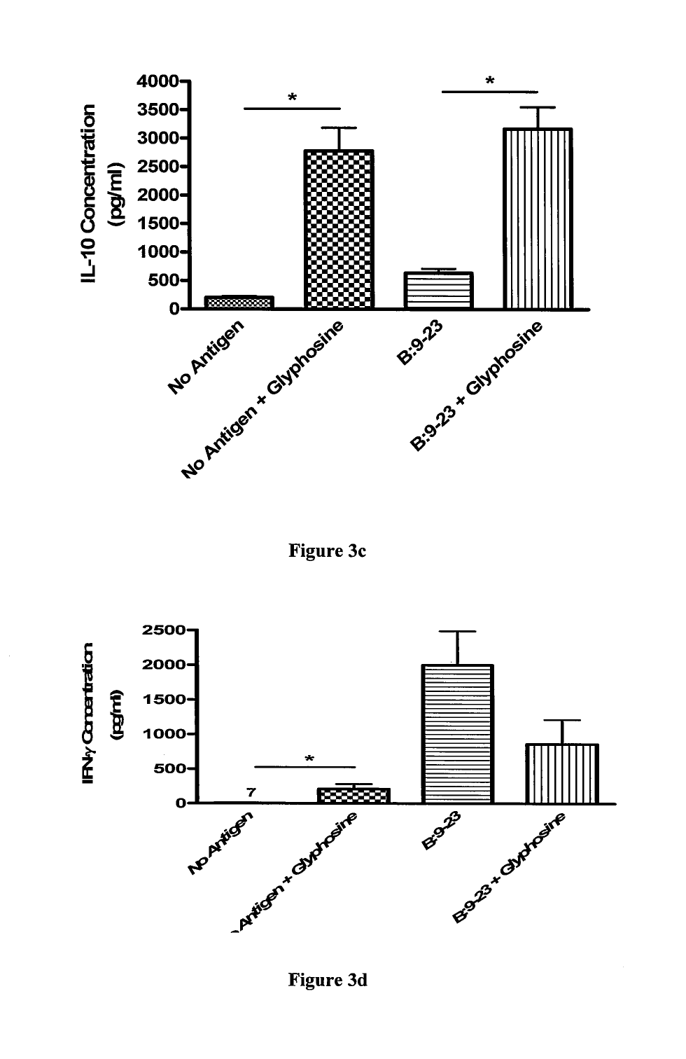

The in vitro response of a monoclonal T cell population targeted to the B:9-23 peptide (rather than hybridomas) was evaluated by enzyme-linked immunospot (ELISPOT). Panel a of FIG. 3 shows results from a transgenic BDC 12-4.1 Rag−/− mouse, expressing a single TCR capable of causing diabetes. As shown in panel b of FIG. 3, glyphosine cultured in vitro with BDC 12-4.1 splenocytes dramatically enhances IL-10 positive cells with a much smaller increase in IFN-γ positive cells. In splenocytes, glyphosine stimulated IL-10 responses both with and without the addition of B:9-23 peptide to their culture. Stimulation without the addition of B:9-23 peptide was unexpected and could be due to in vivo insulin peptide-MHC complexes present in the spleen. As shown in panel d of FIG. 3, both IL-10 (FIG. 3c) and IFN-γ (FIG. 3d) were quantified in the supernatant of stimulated splenocytes. IL-10 levels correlated with the spot numbers from ELISPOT, however, while IFN-γ positive cells are increased with glyphosine absolute levels of IFN-γ are not increased with the addition of glyphosine compared to peptide alone.

IL-10 has anti-inflammatory properties reported to down regulate T helper 1 (Th1) cytokines, such as IFN-γ and TNF-β20, and may upregulate regulatory T cell responses. Coupled with the fact that glyphosine decreases IFN-γ levels in the presence of B:9-23 peptide, glyphosine could be an immunomodulatory agent for the treatment and prevention of type 1 diabetes. Prior to using glyphosine as a therapy, the effects of glysophine upon polyclonal splenocytes from NOD mice were evaluated. As shown in panel e of FIG. 3, a three- to four-fold increase in IL-10 producing cells from NOD splenocytes was found when stimulated with glyphosine and B:9-23 peptide.

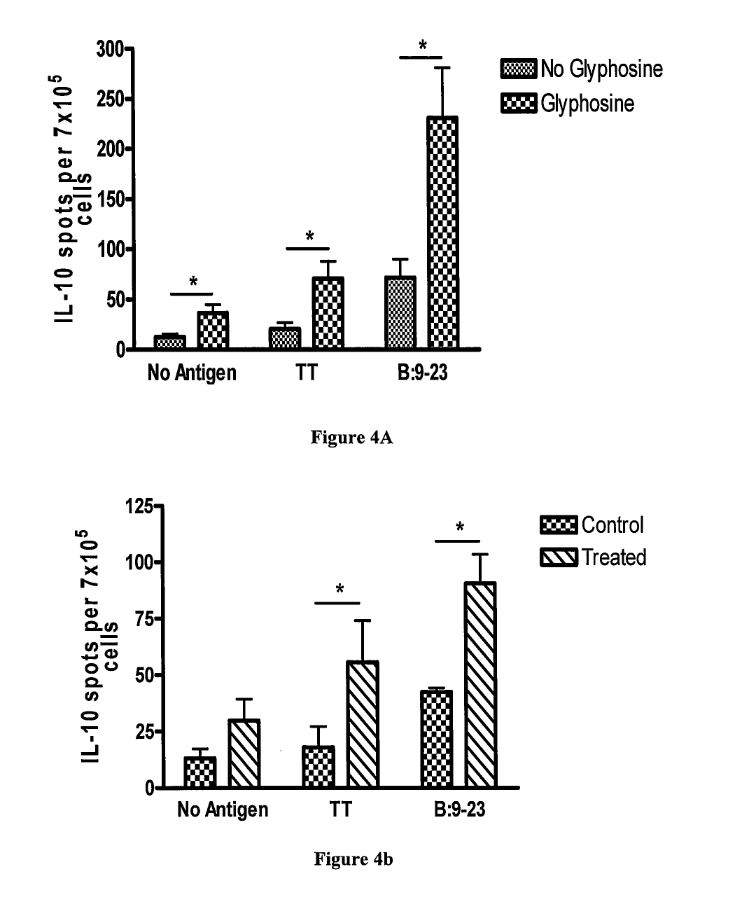

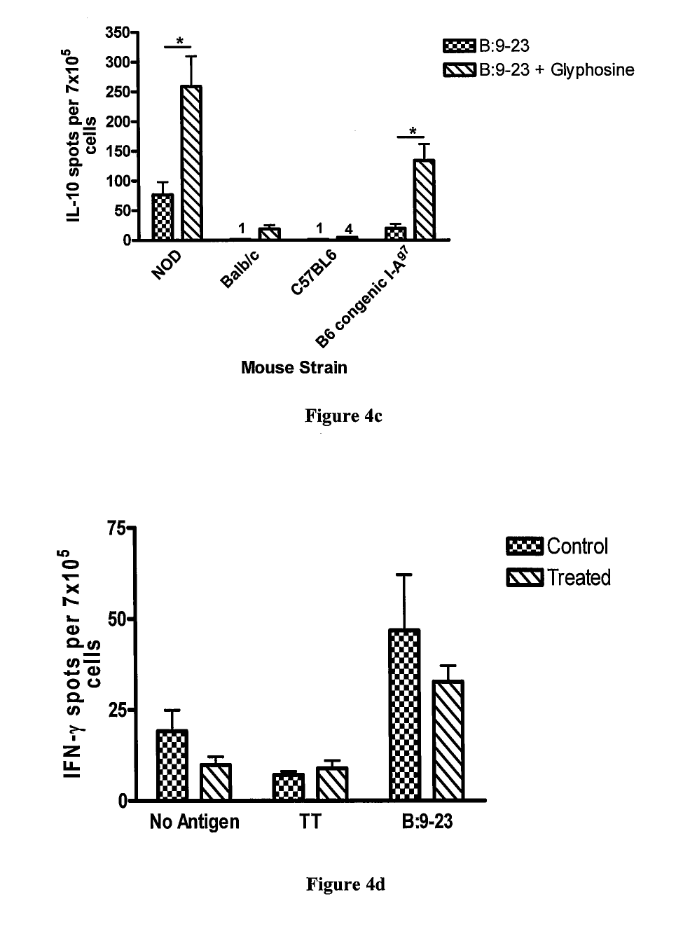

To evaluate the strain specificity of glyphosine plus insulin B:9-23 stimulation, ELISPOT assays were performed using age and sex matched Balb/c and C57BL/6 mice which have different class II presenting molecules from NOD mice. Balb/c mice have I-A^{d} as their class II antigen presenting molecule. I-A^{d} is structurally similar to I-A^{g7} of the NOD, with both strains having identical I-A alpha chains (17 amino acid differences in the I-A beta chains). C57BL/6 mice have I-A^{b} as their class II antigen presenting molecule. Both strains lack the basic pocket 9 in the peptide binding groove. As shown in panel a of FIG. 4, splenocytes from both strains failed to respond to glyphosine plus B:9-23 peptide as compared to NOD mice (FIG. 30. The lack of response in both strains most likely relates to their different class II alleles. To test this hypothesis, splenocytes from C57BL/6 mice congenic for I-A^{g7} were cultured with B:9-23 peptide and glyphosine (FIG. 4, panel b, right columns). The I-A^{g7} congenic splenocytes responded to glyphosine plus B:9-23 peptide stimulation, genetically mapping the IL-10 response to the MHC region.

I-A^{g7} containing splenocytes respond to glyphosine and B:9-23 peptide in vitro and therefore, in vivo stimulation following glyphosine treatment was evaluated. Adult female NOD mice with insulitis but without diabetes received daily injections of glyphosine for 5 days. ELISPOT assays with ex vivo splenocytes cultured with peptides from glyphosine treated and control mice demonstrated upregulation of IL-10 (panel c, FIG. 4) along with no change in IFN-γ (panel d, FIG. 4). Following in vivo administration of glyphosine there was upregulation of IL-10 from splenocytes cultured in vitro with B:9-23, as well as those without B:9-23 peptide added to culture, similar to the transgenic BDC 12-4.1 mice.

### Example 5

### Effects of In Vivo Administration of Glysophine

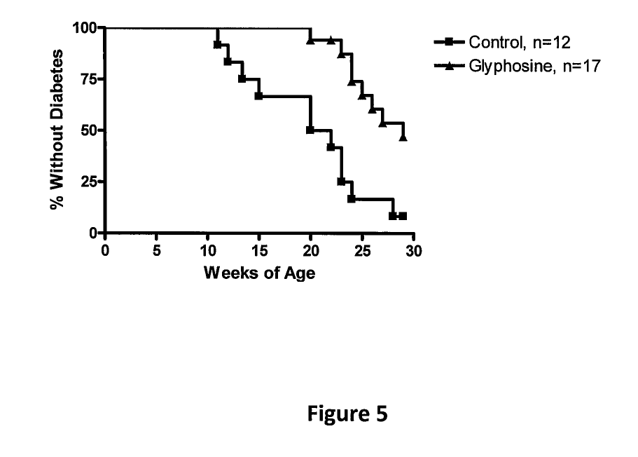

Having demonstrated that in vitro and in vivo glyphosine enhances stimulation of IL-10 secretion by the insulin B:9-23 peptide, in vivo administration was evaluated to for efficacy in delaying development of diabetes (FIG. 5). NOD mice received glyphosine starting at 4 weeks of age and treatment concluded at 21 weeks of age. Prevention of diabetes occurred as long as therapy was administered; however, stopping glyphosine treatment resulted in diabetes development at a rate similar to the control mice. The delayed diabetes development in glyphosine treated mice is statistically significant compared to controls (p<0.001).

These studies are the first to identify small molecules that interact with the MHC class II peptide binding groove and do not act as a catalyst for the purpose of altering the T cell response to a specific target peptide. There is significant homology between the high risk class II alleles DQ8 of humans (DQA1*0301-DQB1*0302) and I-A^{g7} of mouse with both having a similar basic pocket 9, and the amino acid sequences of the murine insulin 2 B:9-23 is identical to human B:9-23. In silico molecular docking of the compounds of the NCI/DTP repository for pocket 9 of DQ8 revealed that seven out of the top 50 scoring compounds for both pocket 9 of I-A^{g7} and DQ8 are identical with glyphosine ranking second for DQ8 and seventh for I-A^{g7} (Table 2), suggesting that small molecules targeted to pocket 9 of I-A^{g7} may similarly bind to DQ8. These results demonstrate that the combination of structure guided virtual screening and the concept that small molecules targeted to specific MHC pockets can be immunomodulatory has broad relevance to the prevention and treatment of autoimmunity, such as diabetes.

TABLE 2
Identical Top 50 Scoring Small Molecules Between DQ8 and I-A^{g7}

| | | Docking | |
|---|---|---|---|
| Compound Name/ | | Score | |
| Chemical Formula/ | | For | Docking Score |
| Molecular Weight/ | | DQ8 | I-A^{g7} |
| NCI Number | Structure | (Rank) | (Rank) |
| Glyphosine N,N-bis(phosphonomethyl) glycine C3H10O6P2 204.0559 407817 | 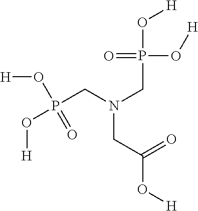 | −42.1 (#2) | −31.1 (#7) |
| Ethane-1,2-diphosphonic acid C2H8O6P2 40837 | 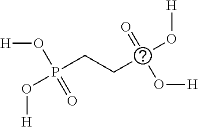 | −37.9 (#4) | −32.0 (#5) |
| 2-methyl-1,1,3- propanetricarboxylic acid C7H10O6 190.1524 25950 | 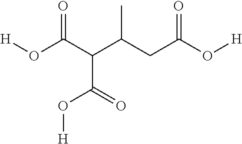 | −36.2 (#7) | −29.2 (#37) |
| 1,1,2- cyclopropanetricarboxylic acid C6H6O6 174.1098 24681 | 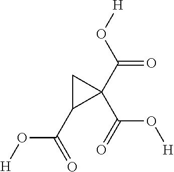 | −35.4 (#10) | −32.2 (#3) |
| 2,3-dihydroxypropyl dihydrogen phosphate C3H9O6P 172.0743 9231 | 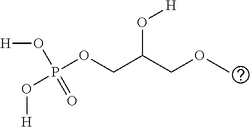 | −33.8 (#25) | −29.5 (#28) |
| 2-sulfinobenzoic acid C7H6O4S 186.182 179320 | 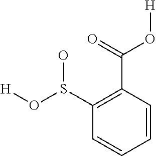 | −33.0 (#42) | −29.6 (#27) |
| 1-amino-1,2,3- propanetricarboxylic acid C6H9NO6 191.1402 206248 | 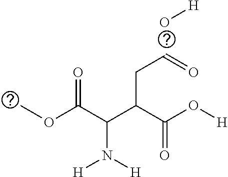 | −32.7 (#46) | −29.4 (#34) |

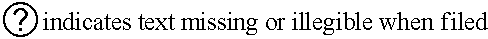

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiments described hereinabove are further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. A method of preventing, treating or ameliorating an autoimmune disease by administering to a mammal in need of such treatment, a therapeutically effective amount of a compound that modifies the T cell response to the targeted antigenic peptide of the autoimmune disease.

2. (canceled)

3. (canceled)

4. The method of claim 1, wherein the compound enhances the binding of an insulin peptide to an MHC class II molecule for presentation to CD4+ T cells.

5. The method of claim 1, wherein the compound is at least one compound selected from the group consisting of:
2,4,6-pyridinetricarboxylic acid,
5-formyl-2-hydroxy-2,4-heptadienedioic acid,
1,1,2-cyclopropanetricarboxylic acid,
3-(hydroxy(oxido)amino)-4-phosphonobenzoic acid,
ethane-1,2-diphosphonic acid,
4-oxo-4H-pyran-2,6-dicarboxylic acid,
N,N-bis(phosphonomethyl)glycine,
N-(2-amino-3-carboxypropanoyl)aspartic acid,
1-hydroxy-15-pyridine-2,6-dicarboxylic acid,
2-(acetyloxy)-5-bromobenzoic acid,
3-C-carboxy-2,4-dideoxy-2-ethylpentaric acid,
4-chloro-2-quinolinecarboxylic acid,
3-methyl-1,2-cyclohexanedicarboxylic acid,
(8-quinolinyloxy)acetic acid,
1-propene-1,2,3-tricarboxylic acid,
3-sulfobenzoic acid,
3-(carboxymethyl)benzoic acid,
phenyl dihydrogen phosphate,
2,3-disulfopropanoic acid,
2-phenylethylphosphonic acid,
3-sulfoisonicotinic acid,
2-(phosphonooxy)benzoic acid,
5,6-dichloro-3-hydroxy-2-pyrazinecarboxylic acid,
3-ethyl-4-oxo-1,2-cyclopentanedicarboxylic acid,
N-(aminocarbonyl)aspartic acid,
2-sulfinobenzoic acid,
2,3-dihydroxypropyl dihydrogen phosphate,
4-chlorophthalic acid, phthalic acid,
2-hydroxy-3,5-diiodobenzoic acid,
3,5-dichloro-2-hydroxybenzenesulfinic acid,
4-(hydroxy(oxido)amino)-3 -methyl-1H-pyrazole-5 -carboxylic acid,
1-amino-1,2,3-propanetricarboxylic acid,
2,6-pyrazinedisulfonic acid,
4-(carboxymethyl)-1H-imidazole-5-carboxylic acid,
2-methyl-1,1,3-propanetricarboxylic acid,
5-chloro-2-hydroxy-3-(hydroxy(oxido)amino)benzoic acid,
1,2-dihydroxy-1,2-ethanedisulfonic acid,
2,5-dibromohexanedioic acid, and
pharmaceutically-acceptable salts thereof.

6. The method of claim 1, wherein the compound is at least one compound selected from the group consisting of:
N,N-bis(phosphonomethyl) glycine,
Ethane-1,2-diphosphonic acid,
2-methyl-1,1,3-propanetricarboxylic acid,
1,1,2-cyclopropanetricarboxylic acid,
2,3-dihydroxypropyl dihydrogen phosphate,
2-sulfinobenzoic acid,
1-amino-1,2,3-propanetricarboxylic acid, and
pharmaceutically acceptable salts thereof.

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. A pharmaceutical composition comprising at least one compound selected from the group consisting of: 2,4,6-pyridinetricarboxylic acid, 5-formyl-2-hydroxy-2,4-heptadienedioic acid, 1,1,2-cyclopropanetricarboxylic acid, 3-(hydroxy(oxido)amino)-4-phosphonobenzoic acid, ethane-1,2-diphosphonic acid, 4-oxo-4H-pyran-2,6-dicarboxylic acid, N,N-bis(phosphonomethyl)glycine, N-(2-amino-3-carboxypropanoyl)aspartic acid, 1-hydroxy-15-pyridine-2,6-dicarboxylic acid, 2-(acetyloxy)-5-bromobenzoic acid, 3-C-carboxy-2,4-dideoxy-2-ethylpentaric acid, 4-chloro-2-quinolinecarboxylic acid, 3-methyl-1,2-cyclohexanedicarboxylic acid, (8-quinolinyloxy) acetic acid, 1-propene-1,2,3-tricarboxylic acid, 3-sulfobenzoic acid, 3-(carboxymethyl)benzoic acid, phenyl dihydrogen phosphate, 2,3-disulfopropanoic acid, 2-phenylethylphosphonic acid, 3-sulfoisonicotinic acid, 2-(phosphonooxy)benzoic acid, 5,6-dichloro-3-hydroxy-2-pyrazinecarboxylic acid, 3-ethyl-4-oxo-1,2-cyclopentanedicarboxylic acid, N-(aminocarbonyl)aspartic acid, 2-sulfinobenzoic acid, 2,3-dihydroxypropyl dihydrogen phosphate, 4-chlorophthalic acid, phthalic acid, 2-hydroxy-3,5-diiodobenzoic acid, 3,5-dichloro-2-hydroxybenzenesulfinic acid, 4-(hydroxy(oxido)amino)-3-methyl-1H-pyrazole-5-carboxylic acid, 1-amino-1,2,3-propanetricarboxylic acid, 2,6-pyrazinedisulfonic acid, 4-(carboxymethyl)-1H-imidazole-5-carboxylic acid, 2-methyl-1,1,3-propanetricarboxylic acid, 5-chloro-2-hydroxy-3-(hydroxy(oxido)amino)benzoic acid, 1,2-dihydroxy-1,2-ethanedisulfonic acid, and pharmaceutically acceptable salts thereof, with at least one pharmaceutically acceptable carrier.

25. (canceled)

26. (canceled)

27. (canceled)

28. (canceled)

29. A method of preventing the development of autoimmune diabetes in a mammal comprising administering to the mammal a pharmaceutical composition comprising at least one compound selected from the group consisting of:
2,4,6-pyridinetricarboxylic acid, 5-formyl-2-hydroxy-2,4-heptadienedioic acid, 1,1,2-cyclopropanetricarboxylic acid, 3-(hydroxy(oxido)amino)-4-phosphonobenzoic acid, ethane-1,2-diphosphonic acid, 4-oxo-4H-pyran-2,6-dicarboxylic acid, N,N-bis(phosphonomethyl)glycine, N-(2-amino-3-carboxypropanoyl)aspartic acid, 1-hydroxy-15-pyridine-2,6-dicarboxylic acid, 2-(acetyloxy)-5-bromobenzoic acid, 3-C-carboxy-2,4-dideoxy-2-ethylpentaric acid, 4-chloro-2-quinolinecarboxylic acid, 3-methyl-1,2-cyclohexanedicarboxylic acid, (8-quinolinyloxy)acetic acid, 1-propene-1,2,3-tricarboxylic acid, 3-sulfobenzoic acid, 3-(carboxymethyl)benzoic acid, phenyl dihydrogen phosphate, 2,3-disulfopropanoic acid, 2-phenylethylphosphonic acid, 3-sulfoisonicotinic acid, 2-(phosphonooxy)benzoic acid, 5,6-dichloro-3-hydroxy-2-pyrazinecarboxylic acid, 3-ethyl-4-oxo-1,2-cyclopentanedicarboxylic acid, N-(aminocarbonyl)aspartic acid, 2-sulfinobenzoic acid, 2,3-dihydroxypropyl dihydrogen phosphate, 4-chlorophthalic acid, phthalic acid, 2-hydroxy-3,5-diiodobenzoic acid, 3,5-dichloro-2-hydroxybenzenesulfinic acid, 4-(hydroxy(oxido)amino)-3-methyl-1H-pyrazole-5-carboxylic acid, 1-amino-1,2,3-propanetricarboxylic acid, 2,6-pyrazinedisulfonic acid, 4-(carboxymethyl)-1H-imidazole-5-carboxylic acid, 2-methyl-1,1,3-propanetricarboxylic acid, 5-chloro-2-hydroxy-3-(hydroxy(oxido)amino)benzoic acid, 1,2-dihydroxy-1,2-ethanedisulfonic acid, and pharmaceutically acceptable salts thereof.

30. (canceled)

31. (canceled)

32. A method of delaying the onset of autoimmune diabetes in a mammal comprising administering to the mammal a pharmaceutical composition comprising at least one compound selected from the group consisting of: N,N-bis(phosphonomethyl) glycine, Ethane-1,2-diphosphonic acid, 2-methyl-1,1,3-propanetricarboxylic acid, 1,1,2-cyclopropanetricarboxylic acid, 2,3-dihydroxypropyl dihydrogen phosphate, 2-sulfinobenzoic acid, 1-amino-1,2,3-propanetricarboxylic acid, and pharmaceutically acceptable salts thereof.
